(19) United States
(12) Patent Application Publication
(10) Pub. No.: US 2026/0325052 A1
(43) Pub. Date: Oct. 22, 2026

(54) RAPID AND PRECISE ASSESSMENT AND TRAINING OF OCCULAR, VESTIBULAR, AND MOTOR BEHAVIOR IN STROKE PATIENTS

(71) Applicant: University of Rochester, Rochester, NY (US)

(72) Inventors: Duje Tadin, Pittsford, NY (US); Emily L Isenstein, Rochester, NY (US); Ania C Busza, Rochester, NY (US)

(73) Assignee: University of Rochester, Rochester, NY (US)

(21) Appl. No.: 19/108,775

(22) PCT Filed: Sep. 11, 2023
(86) PCT No.: PCT/US23/73847

§ 371 Date: Mar. 5, 2025

(51) Int. Cl. A61B 5/00; A61B 5/11; A61B 5/16; G06F 3/01

(52) U.S. Cl. CPC A61B 5/4064; A61B 5/1101; A61B 5/1124; A61B 5/162; A61B 5/163; G06F 3/013

(57) ABSTRACT

A method of rapid assessment of fine motor skills including eye movements, truncal stability, and finger movements using virtual reality and application in cerebellar stroke includes providing a VR device to be worn by a stroke patient; instructing the stroke patient to observe various visual stimuli, tap their fingers, or reach for a VR target without a view of their hand while they reach for the target, forcing a high reliance on proprioception; and evaluating a reaching error in reaching for the target based on a reach accuracy.

### CROSS REFERENCE TO RELATED APPLICATION(S)

This application is a national stage application under 35 U.S.C. § 371 of PCT Application No. PCT/US2023/73847, filed Sep. 11, 2023, RAPID AND PRECISE ASSESSMENT AND TRAINING OF OCCULAR, VESTIBULAR, AND MOTOR BEHAVIOR IN STROKE PATIENTS, published as WO 2024/055032, which claims priority to and the benefit of U.S. provisional patent application Ser. No. 63/405,200, RAPID AND PRECISE ASSESSMENT AND TRAINING OF OCCULAR, VESTIBULAR, AND MOTOR BEHAVIOR IN STROKE PATIENTS filed Sep. 9, 2022 the entire contents of which are incorporated by reference herein.

### TECHNICAL FIELD

The application relates to virtual reality (VR) technology in medical use, and, more particularly, relates to the use of VR technology to assess, diagnose and intervene with in patients with neurological injuries, including stroke.

### BACKGROUND

When a patient presents with an emergent symptom of sudden onset dizziness, clumsiness, or gait instability, the standard of care is to urgently evaluate them for possible stroke. While an MRI and/or CT scan are the gold standard tests for stroke diagnosis, clinicians often conduct a battery of subjective assessments at the bedside to estimate the likelihood that the symptoms are due to stroke and to rule out less urgent conditions such as peripheral vestibular disease. The main advantage of these assessments is that they can be done quickly and the results can be used to make decisions about subsequent assessments and/or treatments. The same assessments can also be used to track stroke recovery because, unlike MRI and CT, they can be performed frequently and at most locations, including hospital rooms and doctor's offices. These assessments can include the “finger-to-nose” test (to assess for tremor and limb coordination), evaluation of truncal stability and ataxia, evaluation of eye movements for nystagmus and disconjugate gaze, and measurement of the speed and regularity of finger tapping. While these assessments have the advantage of quick testing, many are subjective and do not provide precise measurements. This is a problem both for obtaining accurate data for screening and especially for accurately tracking recovery. This can be solved with an approach that retains the speed of subjective assessments but also produces accurate quantitative data in a framework that can be widely and quickly deployed. A virtual finger-to-target assessment has already been published on a group of young adult controls and two patients with recent strokes; further validation has been conducted in 8 additional patients.

### SUMMARY

A battery of rapid assessments that measure fine motor movements associated with cerebellar stroke and other syndromes with motor or vestibular dysfunctions has been developed using virtual reality (VR). Full testing and validation have been done on a method of rapid assessment of hand reaching that includes providing a VR device to be worn by a stroke patient or a suspected stroke patient; instructing the patient to reach for a VR target with or without a view of their own hand while they reach for the target. When the hand is not visible, it forces exclusive reliance on proprioception. Measurement of the reaching error while reaching for the target allows for assessment of reaching accuracy and speed. This assessment uses VR technology to evaluate precise motor control of hand movements with and without corresponding visual input. This controlled addition and removal of visual input allows isolation of the proprioceptive sense. Other assessments in the battery include the assessment of eye movements as different stimuli are presented, measurement of eye movements and nystagmus when various visual stimuli are presented, head stability when sitting or standing, and how rapidly someone is able to tap their fingers.

The method can further include a treatment or intervention by instructing a repeated performance of the steps instructing through evaluating.

The method can further include sounding an auditory cue which serves as a cue to regulate and standardize the timing of assessment tasks.

In some embodiments, a method for assessing a patient, comprises:
positioning a virtual reality (VR) on a patient to create a virtual environment; and
detecting one or more responses of the patient to stimulus introduced within the virtual environment; and
determining whether the patient incurred a medical event based at least in part on the one or more responses.

In some embodiments, the medical event is a neurological event including without limitation a cerebral stroke.

In some embodiments, the method includes instructing the patient to at least one of track or fixate on the stimulus within the virtual environment. In embodiments, detecting one or more responses of the patient may include tracking reaching movement toward the stimulus, the stimulus being one or more virtual objects introduced into the virtual environment. In some embodiments, the tracking of reaching movement toward the one or more virtual objects is performed one or more multiple ways; in a first instance with the patient's hand visible in the virtual environment and in a second instance when the patient's hand is not visible in the virtual environment. In embodiments, determining differences in one or more of precision or accuracy of the tracked reaching movements during the first instance and the second instance.

In one illustrative embodiment, tracking reaching movement toward the stimulus is performed in a first instance when the one or more virtual objects are visible in the virtual environment and in a second instance when the one or more virtual objects are not visible in the virtual environment. In embodiments, determining differences in one or more of precision or accuracy of the tracked reaching movements during the first instance and the second instance.

In some embodiments, detecting one or more responses of the patient includes at least one of tracking eye movement; tracking truncal ataxia and tracking finger speed.

In certain embodiments, quantifying the one or more responses to determine the occurrence of the medical event.

In embodiments a method for assessing a patient, comprises positioning a virtual reality (VR) device on a patient to create a virtual environment and measuring the patient's fine motor movements in one or more responses of the patient to stimulus introduced within the virtual environment to determine whether the patient incurred a medical event.

The medical event is one of a neurological event or a cerebellar stroke.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIGS. 1A and 1B are diagrams illustrating an exemplative task and stimuli associated with a visible/invisible hand experiment that isolates proprioception in accordance with illustrative embodiments of the present disclosure;

FIGS. 2A and 2B are diagrams illustrating an exemplative experiment associated with a memory demand on a reaching task in accordance with illustrative embodiments of the present disclosure;

FIGS. 3A-3D are graphs illustrating results associated with an exemplative visible/invisible hand experiment in healthy adults in accordance with illustrative embodiments of the present disclosure;

FIG. 4 are diagrams illustrating reaching errors for each individual trial in twenty (20) healthy adult participants in the visible/invisible hand experiment in accordance with illustrative embodiments of the present disclosure;

FIGS. 5A-5D are graphs illustrating results associated with an exemplary memory delay experiment in healthy adults in accordance with illustrative embodiments of the present disclosure;

FIG. 6 are diagrams illustrating reaching errors for each individual trial in eighteen (18) healthy adult participants in the memory delay experiment in accordance with illustrative embodiments of the present disclosure;

FIGS. 7A-7D are graphs illustrating results associated with a visible/invisible Hand experiment in patients with recent cerebellar strokes in accordance with illustrative embodiments of the present disclosure;

FIG. 8 is a graph illustrating reaching errors in stroke patients and healthy age-matched adults in accordance with illustrative embodiments of the present disclosure; and

FIG. 9 is a flow chart illustrating a method of assessing a medical event in a patient according to one or more illustrative embodiments of the present disclosure.

### DESCRIPTION

In the description, other than the bolded paragraph numbers, non-bolded square brackets (“( )”) refer to the citations listed hereinbelow. Each of the disclosures of these references are incorporated by their respective entireties by reference herein.

### A. Introduction

Head-mounted virtual reality (VR) provides a multisensory and engaging experience by immersing the user in a 360° computer-generated environment. This technology affords an opportunity to change the way that perception and action research is conducted, bringing the potential for tightly controlled yet naturalistic experiments that can be conducted while the participant is in motion. Historically, action-perception research has generally involved relatively rigid experimental setups where simple stimuli are presented, with participants indicating their perception with a button press. While this framework has led to major functional and mechanistic advances in our understanding of how the brain processes sensory stimuli, it often treats perception as a passive, unidirectional process and belies the complex reciprocity of the action-perception loop (1). These experiments typically employ simple, two-dimensional stimuli and are conducted in quiet, confined spaces by stationary participants to achieve a high degree of experimental control (2).

Further, many studies involving movement tend to be restricted by a small number of reaching target locations (3-5) or the movement is limited to small actions such as pressing a button (6-8). These limitations of typical perception and action experiments are motivating an effort to develop more active, naturalistic experiments (9-14). One or more goals of the present invention includes capturing the dynamic, bidirectional richness and complexity of everyday experiences. The promise of head-mounted VR displays is that they will allow us to conduct much needed naturalistic and interactive studies of human perception while giving up little, if any, of the experimental control that is the cornerstone of empirical perception research. With VR, we can undertake increasingly complex questions about perception while also applying the findings to more diverse populations in real-life contexts. Neuroimaging research has shown that human brains are more attuned to complex, naturalistic stimuli over those that are simple and artificial (15). VR technology can be customized to present three-dimensional images (16-18), create the illusion of distant sounds (19, 20), and provide haptic feedback to create engaging, multimodal stimuli that represent the lived experiences of research participants (21-23). VR can also incorporate a high degree of control in a realistic and multisensory environment, ideal for high quality basic research. For example, a recent study used VR in conjunction with eye-tracking to progressively remove the color from peripheral vision during free-viewing of immersive 360° videos, dramatically revealing the limitations of human color perception in the visual periphery (24). This technology has also been used to assess audiovisual speech perception in children (25) and verticality perception in patients with symptoms of dizziness (26).

VR environments can also be constructed to be responsive to user input, allowing participants to behave closer to how they would in a real-world situation (27-29). This sense of ‘presence,’ which captures the feeling that a user is truly there in virtual world, results from the immersion the user feels as a result of realistic multisensory illusions (30, 31). This feeling also provides a sense of agency over the environment, increases task engagement, and can affect cognition, social behavior, and memory (1, 32, 33). Naturalistic stimuli also capture and maintain attention more authentically than simple two-dimensional stimuli because they tap into more sophisticated top-down attention pathways that incorporate context, prior knowledge, and goals rather than purely feature-based attention (34).

A recent benefit of head-mounted VR lies in its ability to easily capture data from a moving participant, allowing perception and action to be studied simultaneously during active, full-body tasks. As most research on perception is conducted with a stationary participant, this ability to concurrently examine how people physically interact with and respond to their environment provides new opportunities to study the action-perception loop. Of particular importance for the present disclosure, VR headsets are able to accurately track fine motor movements in real time. This includes head motion, the position of the hands, including precise finger movements, and movement of the eyes. One such widely available device, the Meta Quest II (Meta, USA) has <1 cm hand tracking accuracy in well-lit environmental conditions (35). The subsequent release of Meta Quest Pro also added eye tracking as a standard feature. The implications of simple and effortless eye and body tracking technology are considerable; in particular, experiments studying human movement, posture, and proprioception in clinical populations stand to benefit from this technology. Crucially, the portability of VR headsets means that research can occur in places that cannot accommodate traditional lab equipment, such as a hospital room or out in the community. Larger groups of more diverse populations can be evaluated because conditions can be replicated with very high fidelity regardless of the participant's location or circumstances. Commercially available VR headsets are also impressively accessible in terms of cost, portability, and ease of use. As a portable “lab in the box,” a headset has the potential to increase sample sizes, reach under-studied populations, and promote long-distance scientific collaborations.

One area of VR research that has received a great deal of attention is in stroke rehabilitation, with a specific focus on visual-motor coordination and perception. Over 100 randomized control trials have been conducted testing VR technology with people recovering from stroke, with the majority published in the past five years. There is substantial diversity in the attributes of the investigations: studies have been conducted in the home (36-38), in conjunction with telehealth resources (39-41), and in patients with both acute (42-44) and chronic (45-47) stroke. The majority of work on motor rehabilitation only assessed gross motor skills (e.g., reaching) by tracking the position of the handheld controller (44, 48) or tracked finger motion by using supplemental specialty equipment (49, 50). However, persistent fine motor dysfunction is a common consequence of stroke and dramatically affects activities of daily living (51, 52), requiring rehabilitative techniques that target fine motor skills. Hand-tracking technology built into VR offers a promising avenue to examine the speed, accuracy, and consistency of fine motor movements as baseline assessments and/or measures of rehabilitative progress.

To assess the feasibility of using VR technology to study fine motor skills, body movements, and eye movements in both healthy and clinical populations, the present disclosure employs hand-tracking to measure accuracy in simple visual and reaching tasks while varying multisensory and cognitive demands. This study was inspired by previous tasks that used mirrors (53) or tablets (54) to manipulate hand or target visibility during reaching. The approach detailed in the present disclosure is distinct in that it relies on a set of simple, naturalistic VR tasks that do not require complex instructions or training and can be administered quickly while still getting high precision data on fine reaching movements. Two different published experiments have already been conducted with twenty (20) healthy young adults: one assessed visual-proprioceptive integration versus isolated proprioception, and the other tested spatial memory. These two tasks were selected to examine the sensitivity of VR-based reaching assessment under different sensory and cognitive conditions. The visual-proprioceptive task was also completed by individuals with recent cerebellar strokes to evaluate the practicality of successfully collecting this data with individuals with motor or vision difficulties. Additional data has been collected in 8 patients with recent cerebellar stroke, 25 healthy older adults age matched to the patients and 37 young adults. Overall, the goal of these studies was to evaluate whether VR-based hand tracking can serve as a sensitive measure of differences in fine motor movements across various conditions in individuals with and without visuo-motor disabilities. Further study of the use of built-in eye-tracking to measure eye movements, the controller to measure finger tapping speed, and the headset to measure truncal stability also have been performed and continuously analyzed.

### B. Materials and Methods

For two hand reaching experiments, including Experiments 1 and 2, healthy young adult participants were recruited from a local community area. For a third experiment, including Experiment 3, a total of ten patients rehabilitating from cerebellar strokes from a local hospital were recruited. This included two patients whose data have already been published and data from an additional eight patients whose data have been collected since the original publication. Each healthy participant completed the Edinburgh Handedness Inventory (55) and a demographic survey. All participants had normal or corrected to normal hearing and all healthy participants had normal or corrected to normal vision. Written informed consent was obtained from all participants. The follow-up experiments in young adults, healthy older adults and older adults with recent stroke followed the same recruitment and screening protocol.

The virtual reality experiments were conducted using a 1st generation head-mounted Oculus Quest running the latest OS/firmware at the time of testing. UNITY version 2019.4.2f was used to create the experiments. SideQuest, a free 3rd party software, was used with the scrcpy plugin (https://github.com/Genymobile/scrcpy) so experimenters could monitor what the participant saw on the headset during the experiment. Healthy participants were seated in the experiment room on a stationary chair whereas participants with recent stroke conducted the experiment in a stationary chair next to their hospital bed. All experiments were conducted with no objects in front of the participants in rooms with good lighting to optimize the environment for hand-tracking. All participants were given a brief introduction on how to navigate the virtual reality setup. Participants were instructed to keep their shoulders against the back of the chair during the entire experiment and were monitored continuously and given reminders, as necessary. The Oculus Guardian system, intended to prevent actively moving users from exiting the designated ‘safe’ area by providing a visual warning when the user approaches the periphery of the Guardian area, was disabled to avoid disrupting the experiment. All participants were monitored continuously to maintain a safe experience. Participants were told to put the headset on and to adjust the straps so that it was comfortable. Those wearing corrective lenses were able to wear them under the headset. Help was offered if requested. Participants were also shown the inter-pupillary distance slider at the bottom of the headset, and told to move it around until they found their “sweet spot,” where the images/text were clearest and most legible. The inter-pupillary distance on the Quest headset ranges from 58 mm-72 mm. This wide range allowed participants to adjust the lens spacing for a comfortable viewing experience in VR. Similar parameters would be used for assessments using the Meta Quest Pro, which includes eye-tracking and is able to run the experiments that involve measuring eye-movements.

In the hand reaching assessments, once each experiment loaded, participants viewed a grey, featureless room. Instructions appeared directly in front of them, and rendered representations of each of their hands appeared. These hand renderings moved and articulated in real-time corresponding to the participant's real hand movements. Participants were asked to indicate which was their dominant hand; once a hand was selected, only that hand was visible and functional for the remainder of the experiment. To ensure the reaching distance was appropriate to the size and motor function of each individual, participants extended their dominant arm to calibrate the reaching distance before each experiment. The distance from the end of the extended arm to the headset was used as the distance of the radius on which target stimuli would appear. In the original published work, the target appeared along an arc that is equidistant from the midline. Subsequent versions may include having the target appear along an arc that is equidistant from the shoulder of the hand being used in the assessment; this prevents the participant from having to reach further for certain targets.

Each healthy participant completed one practice session and two separate experiments, the Visible/Invisible Hand experiment and the Memory Delay. Stroke patients completed one practice session and only the Visible/Invisible Hand experiment to reduce fatigue and avoid possible confounding cognitive factors in the Memory Delay experiment. In each trial of the practice session, a colored sphere, e.g., a pink sphere (target) appeared along an invisible 60-degree arc at arm's length in front of the participant; the radius of this arc was set by the extended arm in the experiment's introduction and the arc extended indefinitely vertically. Using their dominant hand, participants were instructed to touch the target sphere with their index finger. Each trial ended when the fingertip passed through the arc; the target would then disappear and the next trial would begin regardless of the accuracy of the reach. They were then instructed to move their hand back to touch a cube that appeared just in front of their chest. The cube served as a reset point that appeared once the target sphere disappeared. Once the index finger touched the cube, the cube would disappear and after 500 milliseconds (ms) a new target sphere would appear randomly along the 60-degree arc. The program specifically recorded the difference in degrees between where the tip of the index finger passed through the arc and the center of the target, accounting for both horizontal and vertical error. Participants were encouraged to take breaks by resting their hands on their lap to avoid fatigue. Participants completed practice trials until they felt comfortable with the motions and the experimenter deemed them ready to begin the experiments. Additional visual feedback, including an indication of where the finger passed through the arc so that the participant knows how far from the target they were, may be added in later versions.

The two experimental conditions retained the same basic structure as the practice session, but with two sets of key modifications.

### C. Experiment 1—Visible/invisible Hand

This experiment used the same introduction and structure as the practice session, but in 50% of the trials the rendering of the dominant hand became invisible during the reaching phase. In these invisible hand trials, the participant had no visible feedback on where their hand was while they were reaching for the target, forcing exclusive reliance on proprioception. The hand reappeared only after the reach movement was completed. This is a critical manipulation that isolates proprioceptive function. Each participant completed 10 practice trials and 200 experimental trials. Experimental trials were split into 100 hand visible randomly interspersed with 100 hand invisible trials. Invisible and visible trials are randomly interspersed to prevent participants from adopting different strategies in the two types of trials. In addition, the duration of hand invisibility is short, which, together with randomly interspersed trials, contributes to a smooth experience in which hand invisibility is not subjectively salient.

The experiment took between 5-6 minutes to complete in healthy adults. Data analysis (detailed below) showed that this data collection can be shortened by 75% (~1.5 minutes of data collection) without a significant loss is measurement precision.

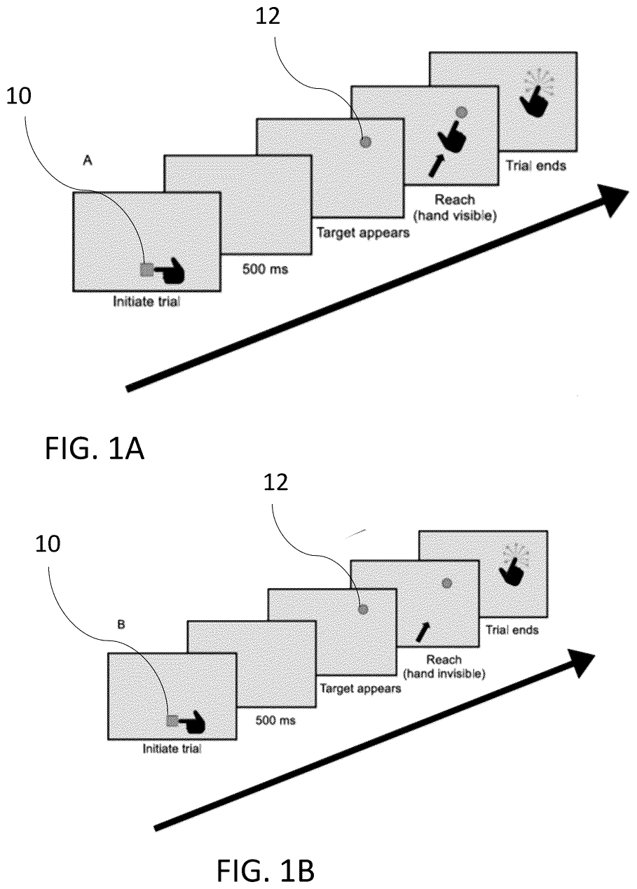

Referring to FIGS. 1A and 1B, a task and stimuli associated with a visible/invisible hand experiment is illustrated. Each trial starts with a first colored cube, e.g., a green cube 10 appearing in front of the participant's chest. After the cube is touched, the cube disappears and a circular target sphere, e.g., a pink sphere 12 appears along a 60-degree arc in front of the participant at arm's length. When participant's index finger passes through the arc, it explodes and the trial ends. A new cube appears to begin the new trial. In the visible hand condition of FIG. 1A, the rendering of the hand is visible during the entire trial. In the invisible hand condition of FIG. 1B, the rendering of the hand is invisible during the reach phase. That is, the hand rendering disappeared when the cube was touched, reappearing only at the completion of the reach movement.

### D. Experiment 2—Memory Delay

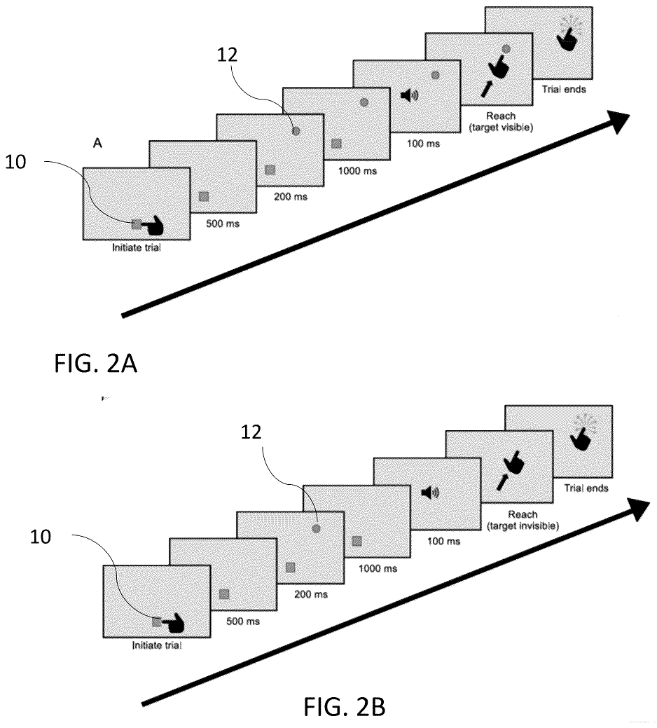

FIGS. 2A and 2B illustrate an experiment associated with a memory demand on a reaching task. This experiment used a similar introduction and structure as the practice session, but in 50% of trials a memory demand on the reaching task was imposed. 500 ms after the participant touched the reset cube, the target would appear and be followed by a tone 1200 ms later. The tone had a frequency of 440 Hz and a duration of 100 ms, and was set at a volume comfortably audible for each individual participant. The tone was presented bilaterally and functioned as a cue for the participant to reach for the target location. In this experiment, the hand remained visible for the entire duration of the experiment. The critical manipulation was the visibility of the target before the reach. In 50% of the trials the target sphere would remain visible for the entire duration of the trial as depicted in FIG. 2A. In the remaining 50% of the trials, the target sphere would only appear for 200 ms then disappear for the remaining 1000 ms before the tone and remain invisible during the subsequent reach movement (FIG. 2B), requiring the use of spatial memory to guide the reach. This approach mirrors established memory-guided reaching tasks by introducing a one second delay (56, 57). As in Experiment 1, participants completed 10 practice trials and 200 experimental trials. The program randomly interspersed the 100 trials in which the target sphere remained visible and the 100 trials in which the target sphere disappeared. The experiment took 8-9 minutes to complete.

### E. Task and Stimuli in the Memory Delay Experiment

In embodiments illustrated in FIGS. 2A and 2B, each trial starts with a colored cube, e.g., a green cube, appearing in front of the participant's chest. 500 ms after the cube is touched, a colored target sphere, e g., a pink sphere appears along a 60-degree arc at arm's length. 1200 ms later, a tone indicates that a participant is free to reach out to the target. When participant's index finger passes through the arc, it explodes and the trial ends. A new cube appears to begin the new trial. In the standard condition of FIG. 2A, the target remained visible for the entire test. In the memory delay condition of FIG. 2B, the target disappeared 200 ms after its appearance, remaining invisible for the 1000 ms before the tone was played and during the subsequent reach movement.

### F. Experiment 3—Visible/Invisible Hand After Cerebellar Stroke

This experiment was identical to Experiment 1, except that the participants included multiple, e.g., two, patients with recent cerebellar stroke. The only difference was that patients took between 15 and 20 minutes to complete the experiment. Also reported are data from 8 additional patients collected since the original publication.

### G. Statistical Analysis

All experiments measured reaching accuracy of the dominant index finger by calculating the difference in degrees between the center of the target sphere and where the tip of the index finger passed through any point along the 60-degree arc where the target could appear. This accuracy was compared between the two conditions of each experiment. In addition, each individual's precision was calculated as the standard deviation of their endpoint accuracy in Experiments 1 and 2. In Experiments 1 and 3, the reaching time-defined as the amount of time between when the target appeared and when the participant's index finger crossed the arc-was also recorded. In all experiments, reaching accuracy was the main outcome measure as it has the greatest potential clinical significance and effect on quality of life and independence. In addition, this approach also includes the ability to track finger position/movement during the entire reaching movement to measure accuracy, tremor, dysmetria, movement delay or reveal evidence of fatigue.

Statistical testing was done with SPSS software version 28 (IBM Corp, Armonk, NY, USA) or MATLAB 2021a software (Mathworks, Natick, MA, USA). Shapiro-Wilk tests of normality were conducted on reaching time, accuracy, and precision in each condition in all experiments, with one or more conditions in each experiment determined as non-normally distributed. Related-Samples Wilcoxon Signed Rank Tests were used in experiments 1 and 2, as statistics were assessed on a group level. In Experiment 3, 294 Independent Samples Mann-Whitney U Tests were conducted because statistics were assessed on an individual level. In Experiments 1 and 3, outliers of >3 standard deviations away from each individual's mean were removed from the reaching time data. In Experiment 1, an average of 2.05±1.00 outlier trials in the visible condition and 2.30±1.26 trials in the invisible condition were removed per participant. In Experiment 3, 7 outlier trials in the visible condition and 2 in the invisible condition were removed form patient 1, and 6 outlier trials in the visible condition and 8 in the invisible condition were removed for patient 2. In all three experiments, reaching accuracy was also assessed including data from only the first 25 trials to evaluate whether our approach is sensitive enough to detect the main results in substantially abbreviated versions of our experiments. Slopes of the change in reaching accuracy over time across conditions were normal across experiments; one sample t tests were conducted to assess whether the slope of the average error differed from zero. No power analyses were conducted prior to data collection because no suitable previous work was available to estimate the sample size needed.

### H. Results

Twenty participants, 8 male and 12 female, participated in Experiment 1, with a mean age of 23.4 (st. dev.=2.6). Eighteen of these participants, 8 male and 10 female, also participated in Experiment 2, with a mean age of 23.6 (st. dev. =2.7). All 38 participants were right-handed, and reported no developmental or psychiatric disorders.

### I. Experiment 1—Visible/Invisible Hand

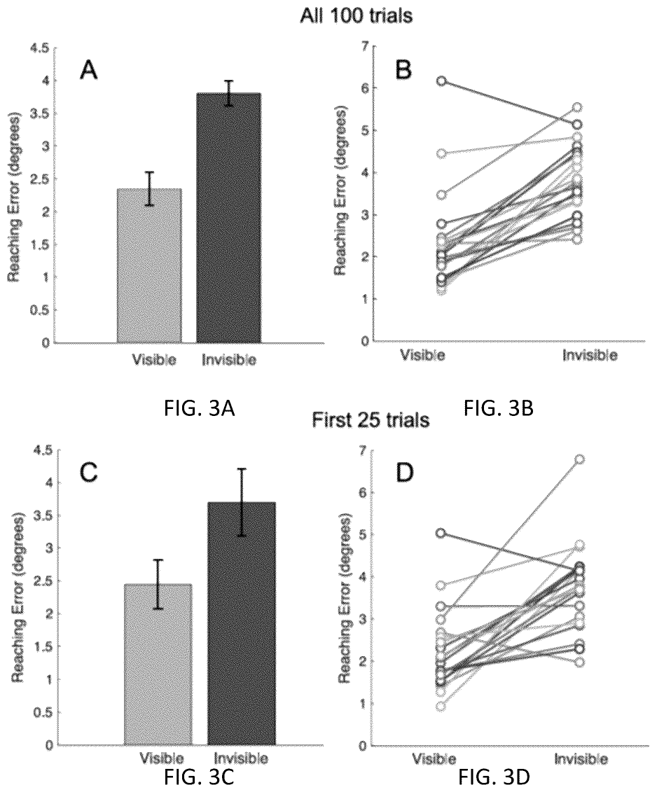

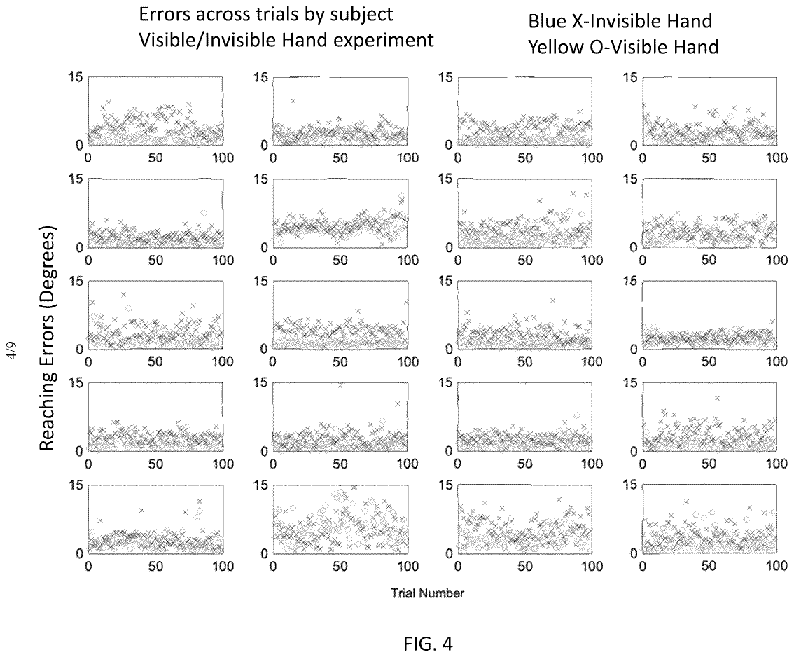

The virtual hand experiment elucidated a clear, robust difference in the reaching accuracy when the virtual rendering of the hand was visible compared to when it was invisible (FIGS. 3A, 3B). We found a significant difference between the average reaching error in visible (2.24°±0.25°) and invisible (3.80°±0.19°) hand conditions (T=204.00, z=3.70, p<0.001; FIG. 3A). This difference was observed in a large majority of individual participants (FIG. 3B). There was also a significant difference between the average reaching precision in visible (1.58°±0.76°) and invisible (1.93°±0.69°) hand conditions (T=160.00, z=2.05, p=0.04). Precision and accuracy were shown to be positively correlated for both the visible (r(18)=0.708, p<0.01) and invisible (r(18)=0.49, p=0.02) hand conditions. There was no significant difference between the average reaching times in visible (625 ms±105 ms) and invisible (617 ms±160 ms) hand conditions (T=87.00, z=−0.67, p=0.50). To determine the sensitivity of this experiment at capturing differences in reaching accuracy, we repeated these statistical tests with only the first 25 trials of each condition. The difference between the visible (2.44°±0.37°) and invisible (3.39°±0.52°) hand reaching accuracy remained significant (T=199.00, z=3.51, p<0.001). This finding, displayed in FIGS. 3C and 3D, confirms that the length of this experiment could be reduced to a fraction of the original length and still provide the same dependable, highly significant result in healthy adults. This is critical for applications in healthcare settings. Participant level data is shown in FIG. 4 to demonstrate the robust consistency of this data across participants and across the duration of the experiment.

In embodiments, FIGS. 3A-3D illustrate results of the visible/invisible hand experiment in healthy adults. In FIG. 3A, group-level average reaching error as a function of hand visibility in all 100 trials. Left vertical bar (Yellow:) visible-hand condition. Right vertical bar (Blue): invisible hand condition. Error bars denote the standard error of the mean. In FIG. 3B, results for 20 individual participants as a function of hand visibility in all 100 trials. In FIG. 3C, group-level average reaching error as a function of hand visibility in the first 25 trials. In FIG. 3D, results for 20 individual participants as a function of hand visibility in the first 25 trials.

FIG. 4 illustrates reaching errors for each individual trial in 20 healthy adult participants in the visible/invisible hand experiment. This depiction of the data allows for visualization of data stability over the course of the experiment. Circle shape (Yellow): visible-hand condition; X shape (Blue): invisible-hand condition. As apparent from the charts/graphs depicted in FIG. 4 reaching errors increase in the absence of a visible hand in the VR environment.

To measure the stability of task performance over time and detect possible learning or fatigue effects, we assessed whether reaching accuracy results in either condition changed throughout the course of the experiment. On a group level, the slope of the average error was not significantly different from zero in both the visible hand (m=0.002, std dev=0.01, t19=0.93, p=0.36) and the invisible hand condition (m=−0.0005, std dev=0.01, t19=−0.27, p=0.79). Evidently, performance remained steady over the course of the full experiment, implying that there was no measurable learning or fatigue effects.

### J. Experiment 2—Memory Delay

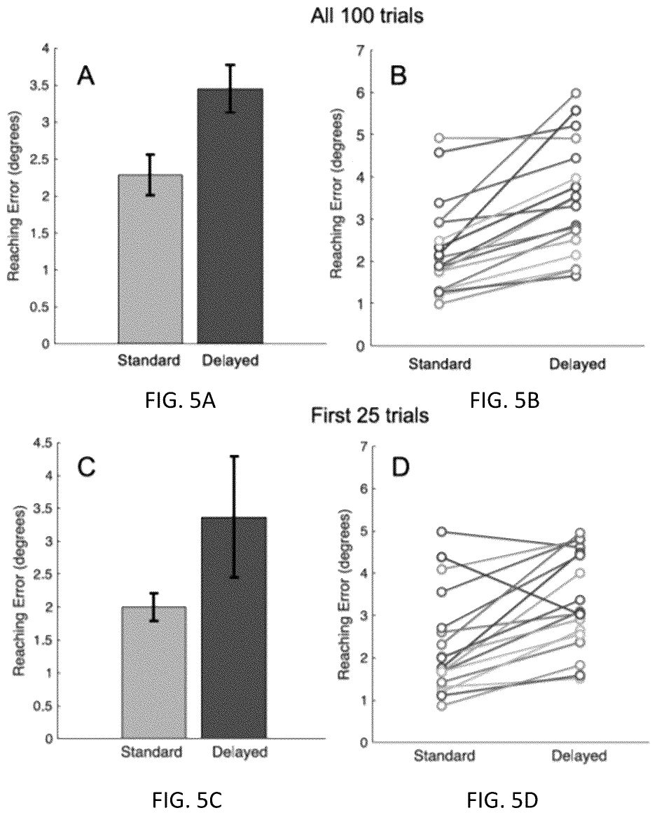

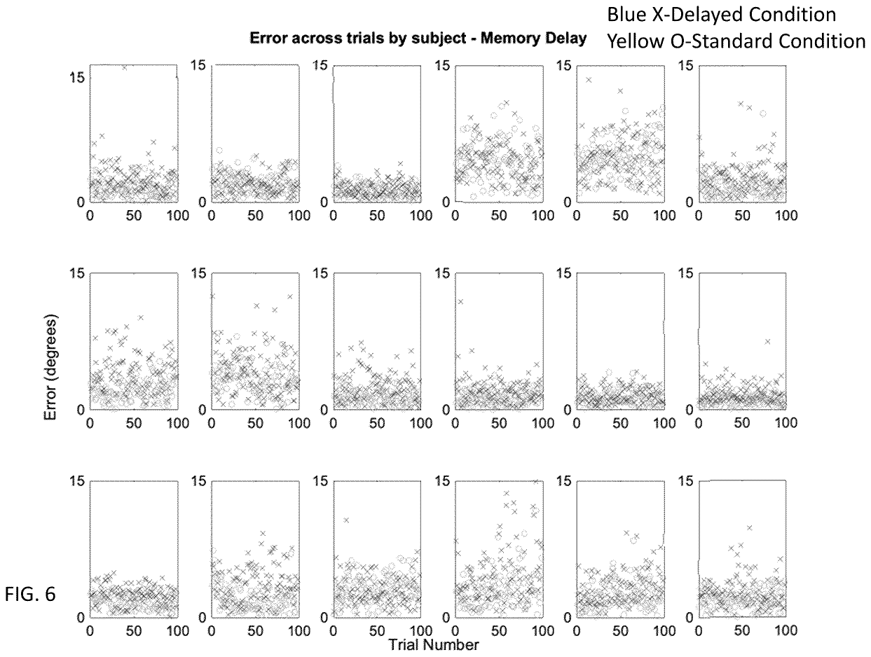

The results of the Memory Delay experiment followed the same pattern as the Visible/Invisible Hand experiment, though results were slightly less robust. We found a significant difference between the average reaching accuracy error in the non-delayed standard condition (2.28°±0.27°) and delayed target condition (3.45°±0.32°; (T=170.00, z=3.68, p<0.001; FIG. 5A). Individual participant data is shown both as averages (FIG. 5B) and with all trials shown (FIG. 6). There was a significant difference between the average reaching precision in standard (1.47°±0.70°) and delayed target (3.36°±3.54°) conditions (T=155.00, z=3.03, p<0.01). Precision and accuracy were shown to be positively correlated for both the standard (r(16)=0.48, p=0.04) and the delayed (r(16)=0.69, p<376.01) conditions.

Additional testing including only the first 25 trials continued to yield significant differences between the standard (2.00°±0.21°) and delayed (3.37°±0.92°) target conditions with respect to reaching accuracy (T=158.00, z=3.16, p<0.01). FIGS. 5C and 5D demonstrate this robust finding after only a quarter of the total trials, affirming that the length of the total experiment could be substantially shorter the original and still reliably distinguish between trial conditions.

In embodiments, FIGS. 5A-5D illustrate results of the memory delay experiment in healthy adults. FIG. 5A illustrates group-level average reaching error as a function of memory demand in all 100 trials depicting a non-delayed standard condition and a delayed condition. Error bars denote the standard error of the mean. FIG. 5B illustrates results for 18 individual participants as a function of memory demand in all 100 trials. FIG. 5C illustrates group-level average reaching error as a function of memory demand in the first 25 trials. FIG. 5D illustrates results for 18 individual participants as a function of memory demand the first 25 trials.

We tested whether reaching accuracy in the two conditions changed over the course of the experiment to evaluate whether there were any learning or fatigue effects. On a group level, the slope of the average error was not significantly different from zero in both the standard (m=0.0019, std dev=0.01, t17=−0.006, p=0.464) and delayed condition (m=0.000674, std dev=0.02, t17=0.45, p=0.773). Thus, as with the first experiment, there were no significant changes in accuracy over time.

FIG. 6 are diagrams illustrating reaching errors for each individual trial in eighteen (18) healthy adult participants in the memory delay experiment in accordance with illustrative embodiments of the present disclosure.

### K. Experiment 3—Visible/Invisible Hand after cerebellar stroke

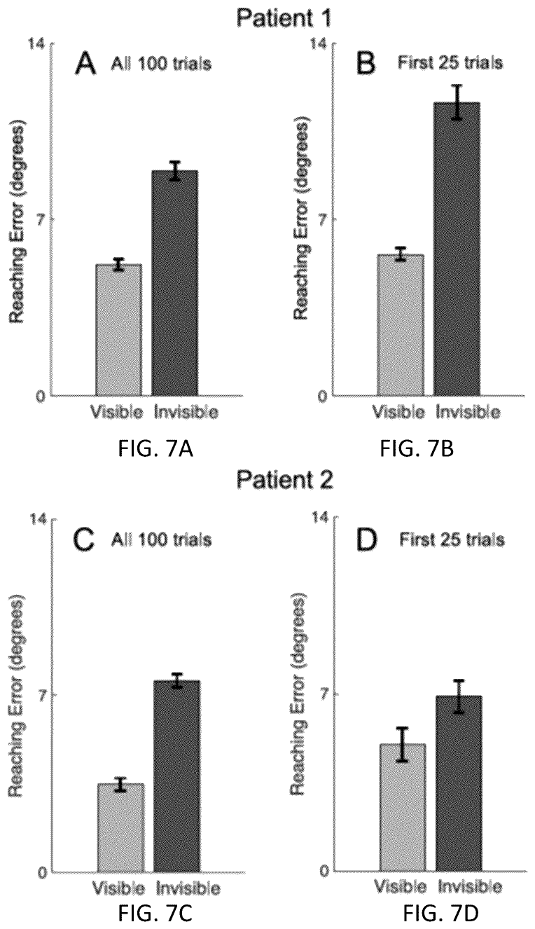

With reference to FIGS. 7A-7D, we focused on the Visible/Invisible Hand experiment in patients with recent cerebellar strokes because the multisensory visual-proprioceptive interaction emphasizes body coordination, which is often affected by stroke (52). This also minimized testing burden for the patients, who completed the experiment with their affected hands. In both patients, we found clear differentiation of reaching accuracy with and without assistance of vision (FIG. 7A, FIG. 7C). Significant differences between the average reaching error in the visible (patient 1:5.23°±2.17; patient 2:3.49°±2.41°) and invisible (patient 1:8.94°±3.47; patient 2:7.56°±2.60°) hand conditions were found on an individual level: patient U(Nvisible=99, Ninvisible=99)=3872.00, z=−2.55, p=0.01; patient 2 U(Nvisible=99, Ninvisible=99)=8053.00, z=7.82, p<0.001. There were significant differences between the average reaching times in visible (patient 1:1781±1270 ms; patient 2: 4339±6066) and invisible (patient 1: 1475±916 ms; patient 2: 2922±2145 ms) hand conditions (patient 1: U(Nvisible=94, Ninvisible=98)=3724.50, z=−2.29, p=0.02); patient 2: U(Nvisible=95, Ninvisible=92)=3615.000, z=−2.04, p=0.04). We again assessed reaching accuracy after only 25 trials for each individual patient. The difference between the visible (patient 1: 5.63°±1.21; patient 2: 4.98°±3.23°) and invisible (patient 1: 11.66°±3.39° patient 2: 6.89°±3.18°) hand reaching accuracy was significant: patient 1 U(Nvisible=25, Ninvisible =25)=605.00, z=5.68, p<0.001; patient 2 U(Nvisible=25, Ninvisible=25)=418.00, z=2.05, p=0.04 (FIG. 5B, FIG. 5D). In the illustrative embodiments, results of the visible/invisible hand experiment in patients with recent cerebellar strokes are depicted in FIGS. 7A-7D. FIG. 7A illustrates reaching error as a function of hand visibility in all 100 trials in patient 1 with a non-delayed standard condition and a delayed condition. Error bars denote the standard error of the mean. FIG. 7B illustrates reaching error as a function of hand visibility in the first 25 trials in patient 1. FIG. 7C illustrates reaching error as a function of hand visibility in all 100 trials in patient 2. FIG. 7D illustrates reaching error as a function of hand visibility in the first 25 trials in patient.

We have conducted an additional, yet unpublished, experiment comparing stroke patients to age-matched healthy controls; this data includes the original 2 patients and 8 additional patients. This data showed significantly higher reaching error in the invisible hand conditions compared to the visible hand conditions, in both the stroke patients and the older adult controls. Notably, there was no significant difference in the average reaching error between groups in the visible hand condition. However, the patient group had significantly larger error in the invisible hand condition as compared to the older adult controls, leading to a nearly two-fold difference in performance. These data highlight the critical importance of isolating proprioception. When the hand was visible, these patients were able to rely on their visual perception to achieve normal reaching accuracy. Their reaching deficit only became apparent when proprioception was isolated using our procedure.

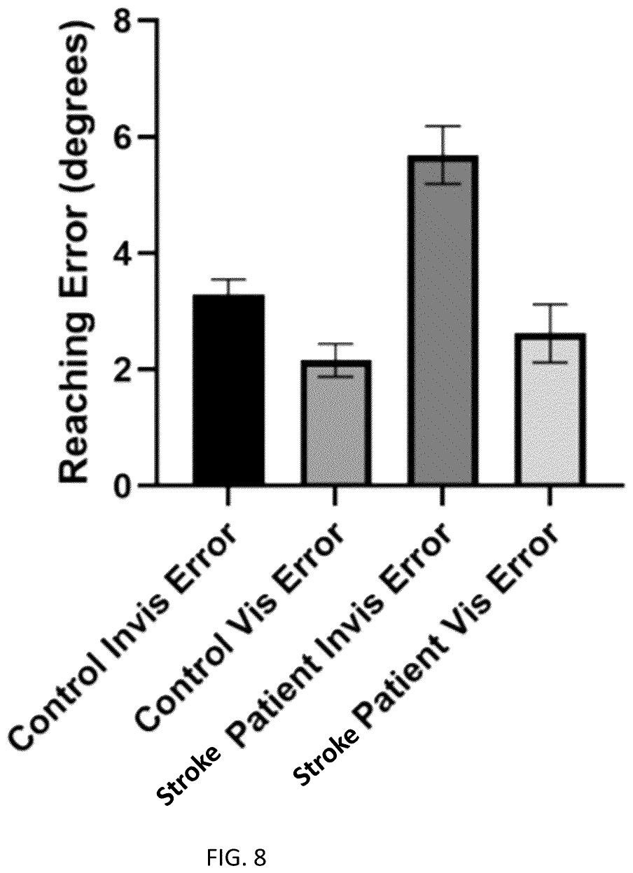

FIG. 8 is a graph illustrating reaching errors in healthy “control” participants and “stroke” patients in accordance with illustrative embodiments of the present disclosure. Of note, in accordance with some embodiments, stroke patients offer substantially similar results to the healthy “control” participants when the hand is visible in the VR environment. However, there is a substantial difference in results in an invisible hand VR environment, i.e., when proprioception is isolated using our approach, showing that isolation is one critical criterion in evaluating stroke occurrence.

### L. Discussion

Our results provide evidence for the utility of built-in hand tracking in head-mounted VR equipment to quickly capture precise information about reaching accuracy. We were able to establish a significant faciliatory effect of vision on reaching accuracy (FIGS. 3A-3D) and demonstrate that adding memory demands impairs reaching accuracy (FIGS. 5A-5D). Our findings that people reach more accurately and precisely, though not more quickly, toward a point when they can see their hand and when the target is visible are not surprising.

They confirm earlier data that vision improves accuracy and precision during reaching (58, 59) and that reaching accuracy and precision deteriorate when memory is required to locate the target (60, 61). Rather, the novelty of the methods of the present invention lies in the manipulation of the sensory experience beyond what is possible in physical reality while collecting robust, consistent data anywhere in a matter of minutes. The main advance here is the ability to quickly and accurately quantify this effect using an approach that isolates proprioceptive function.

By controlling the visual feedback provided by the hand rendering, we are able to uncouple vision and proprioception in the Visible/Invisible Hand experiment, offering a window into how these sensory modalities interact. Typically vision and proprioception are difficult to tease apart without the use of complex equipment such as mirrors (62) and robotics (63), but the use of this new VR technology allows for easy and modifiable adaptations. For example, instead of removing the visual representation of the hand, the rendering of the hand could instead be delayed or shifted to a different location to measure how these changes influence the weighting of visual and proprioceptive information. This weighting remains poorly understood in various clinical populations such as cerebral palsy (64, 65), Parkinson's disease (66, 67), and autism spectrum disorder (68, 69)—that will benefit from research that can isolate and analyze the contributions of each sense and how they change over time.

By introducing a delay and requiring the participants to conduct their reaching movements based on recall, the Memory Delay experiment further assesses reaching in circumstances that require greater cognitive resources. While the delay in this paradigm was relatively short at 1 second, it still has a clear effect on the reaching accuracy. While this effect of memory is expected, our approach offers a way to investigate the spatial representation of memory in a three-dimensional setting. The environment can remain tightly controlled while objects are manipulated, allowing for structured and replicable assessments of spatial memory and navigation. Populations such as older adults and people with recent traumatic brain injury will benefit from further research on the interaction between memory and the ability to navigate a three-dimensional space (70, 71).

Our study also contributes to decades of research confirming benefits when multisensory information is available in domains as varied as memory (72), learning (73), and reaction time (74). In validating the use of VR to study multisensory processes, this new technique provides the capacity to expand on these traditional paradigms to evaluate participants as they move interactively with their environment. Overall, this approach allows for the measurement of action-perception data in a multisensory, naturalistic setting that can be adapted to mimic a variety of real-life scenarios better than the simple and predictable conditions typically found in the lab.

Critically, these experiments also show that VR can be used to efficiently and effectively measure reaching accuracy not only in healthy individuals, but also in those with vision or motor disabilities caused by conditions such as cerebellar infarct. These findings also indicate promise in using this technology to measure parameters relevant to stroke, such as eye and body movements, accurately and quickly. The self-paced nature of these experiments means that they can be adapted to suit individuals with limited mobility, and the ability to adjust the inter-pupillary distance and head position allows for reasonable correction of minor visual issues, as done with the first patient's diplopia. These features allow for the collection of baseline information on post-stroke gross and fine motor skills at a very early stage of recovery and provide the opportunity to potentially distinguish between the effects of ocular and cerebellar issues. Of note, both the results with healthy young adults and those with patients were found to be significant after only a fraction of the trials, indicating that the task could be substantially shortened and still provide a sufficiently precise measure of reaching accuracy. This rapid pace is particularly significant in the context of individuals with muscle weakness who may not be able to sustain activity for long periods of time.

Our results also show that even over a limited number of trials individuals with recent stroke demonstrate changes in their reaching accuracy, suggesting that this paradigm is sensitive to improvement or deterioration, critical for use in rehabilitative training. Of note, we detected a dissociation between the amount of fatigue in the isolated proprioception trials and the visual-proprioceptive integration trials in one of the stroke patients. The ability to measure these differences offers exciting opportunities to learn more about how specific sensory properties are affected by stroke. Moreover, the back-and-forth reaching design of our experiments mimics a clinical evaluation of motor coordination called the finger-to-nose test. By evaluating a patient's ability to quickly and accurately reach for both an externally-referenced target (the administrator's finger) and a self-referenced target (the patient's nose), this clinical test serves as rapid yet imprecise way to measure coordination. Many clinicians use the finger-to-nose test to measure upper-body coordination over the course of stroke recovery (75, 76), but it remains a subjective tool with limited external validity. Using our VR paradigm, these same fine motor skills can be assessed in a way that provides detailed measurements without the need of a trained clinician to administer a coordination assessment.

In illustrative embodiments of the present invention, VR is sensitive, adaptable, can be used by individuals with a variety of limitations, and can be conducted at the bedside.

As mentioned hereinabove, in illustrative embodiments, the present invention leverages VR technology in other stroke assessment methodologies in addition to the assessment of motor control of hand movements described above. Such assessment modalities include, without limitation, a.) assessment of truncal stability/truncal ataxia, b) head impulse-nystagmus-Test of Skew (HINTS) Assessment, c) Visual/Proprioceptive Accuracy (which is a slight adaptation of the hand motor control assessment described hereinabove with the changes being the alignment of the target to the shoulder rather than the midline and the inclusion of visual feedback on accuracy and d) dysdiadochokinesia.

With regard to the a) assessment of truncal stability/truncal ataxia, in embodiments, the following processes may be followed: The seated participant will put on the VR headset and the program is started. The participant is instructed to sit as still as possible. A virtual visual fixation point will be presented to the participant directly in front of them and they will be asked to fixate on the point for a period of time. The time period being recorded will be indicated to the participant. During this period, the headset will measure the small movements of the head as a proxy measure of truncal stability. This protocol may be repeated, however in the second version the headset will not provide any visual stimuli so it will be as if the participant's eyes are closed. The above protocol may be repeated again while the participant is standing rather than sitting. These measurements of truncal stability will be analyzed for the level of truncal ataxia. In embodiments, detected changes in truncal stability may be indicative of an occurrence of a neurological event, e.g., a stroke.

With regard to the b) Head Impulse-Nystagmus-Test of Skew (HINTS) Assessment, in embodiments, the following processes may be followed.
1. Head Impulse—The seated participant will put on the headset and the program is started. A virtual scene will be presented to the participant, with a specific fixation point presented somewhere in the scene. The participant will be instructed to maintain fixation on the point, even if it moves. Eye corrective movements will be measured by either having the virtual scene suddenly rotate in the horizontal plane a certain number of degrees around the participant, as if the participant's head as suddenly turned in one horizontal direction or by having the headset and head rotated by external examiner. The participant's eye movements will be tracked in the x, y, and z planes as they re-fixate on the point.
2. Nystagmus—The seated participant will put on the headset and the program is started. The participant will be instructed to use their eyes to follow a target without turning their head. A target will be presented that will move around the virtual space directly in front of the participant. The target may move smoothly from location to location and pause in certain locations, or may disappear and reappear. The participant's eye movements will be measured in the x, y, and z planes as they follow a smoothly moving target or saccade from the disappearing target to the appearing target. The eye movements may also be evaluated for any torsional (rotational) movement of the eyeball.
3. Test of Skew-The seated participant will put on the headset and the program is started. The participant will be instructed to fixate on a point directly in front of them. The visual stimuli presented to each individual eye will be removed, such that virtual stimuli are only being presented to the other eye. This is as if on eye is being occluded while the other can see. The right and left eye will alternate between which is occluded and which is able to see the fixation point.
4. In each of these tasks, the pattern of eye movements will be assessed to identify presence of abnormal eye movements including nystagmus, breakdown of smooth pursuit, and deconjugate eye alignment. These patterns may be analyzed and used in assessing the occurrence of a neurological event, e.g., a stroke or the like.

With regard to the c) Visual/Proprioceptive Accuracy, in embodiments, the following processes may be followed. The seated participant will put on the headset and the program is started. The participant will see virtual renderings of their left and right hands and will be instructed to use their left or right hand to select which hand they will conduct the test with. The participant is instructed to reach out the selected hand straight out from their shoulder, then pinch their fingers to select a reaching distance. Target 1 will then appear randomly in a degree arc with a radius of the set distance, centered at the x coordinate of the shoulder. Target 2 will always be in the same location just in front of the shoulder. The participant will be instructed to use the index finger on the selected hand to alternate between touching Target and Target 2. Target 2 appears once the finger has reached the distance of the radius, which triggers an explosion at the fingertip. Target 1 appears after Target 2 has been touched. Certain parameters may be manipulated, such as the virtual rendering of the hand may be removed on certain trials so the participant cannot see where their hand is, or Target 1 may be presented on a delay after Target 2 is touched. The path of the hand movements and the accuracy of the reaching for the target will be recorded. In embodiments, finger movements will be tracked throughout the assessment to measure accuracy, tremor, dysmetria, and delay or fatigue, and utilized in potentially identifying the occurrence of a neurological event.

With regard to the d) dysdiadochokinesia, in embodiments, the following processes may be followed: The seated participant will put on the headset and the program is started. The controllers associated with the headset will be place in the participant's hands. A visual fixation point will be presented to the participant directly in front of them and they will be asked to fixate on the point. The participant will be instructed to tap a specific button on the controller using one hand as quickly as possible for 30 seconds. The participant will then complete the same task with the alternate hand. The time point of each button press will be tracked. We will assess how quickly and regularly can the person tap their fingers with both hands.

For all of the above assessments, the assessments may be performed individually for diagnostic and potentially therapeutic purposes (for example as part of rehabilitation exercises), or may be performed as a battery of tests to measure the probability of current symptoms being related to stroke. Assessments may be done with one or both hands. Change of performance over multiple sessions may be assessed, as a measurement of relative improvement or worsening of function. Change of performance over a single session may be assessed, as a measurement of learning or fatigue.

In embodiments, a method of rapid assessment of eye and hand reaching movements using virtual reality and application in cerebellar stroke comprises providing a VR device to be worn by a stroke patient; instructing the stroke patient to track or fixate on targets with their eyes, passively measure truncal stability, measure the speed and regularity of finger tapping, reach for a VR target without a view of their own hand while reaching for the target, forcing a high reliance on proprioception; and evaluating a reaching error in reaching for the target based on a reach accuracy. The method may further comprise a treatment or intervention by instructing a repeated performance of the steps instructing through evaluating. The method may further comprise sounding an auditory cue which serves as a cue for the participant to reach for the target location.

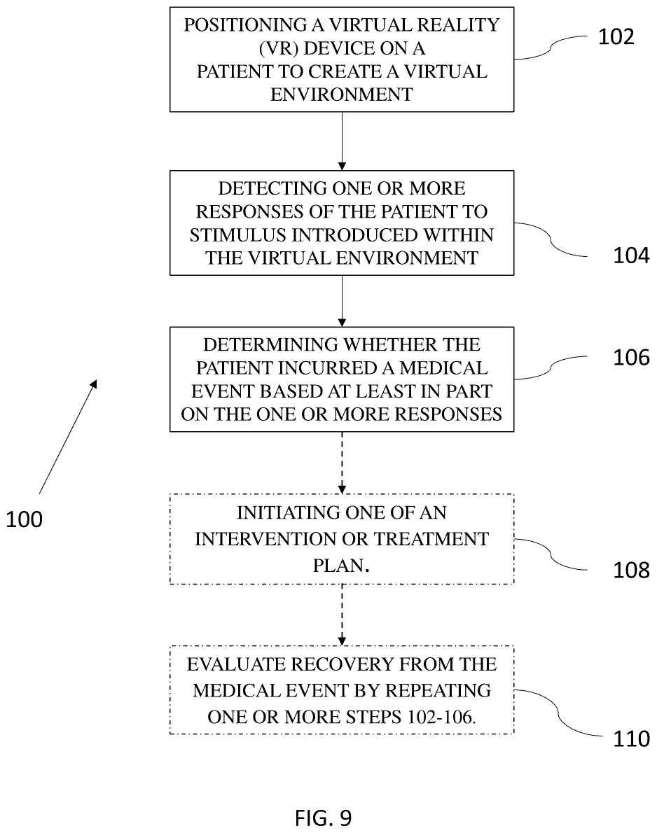

FIG. 9 is a flow chart illustrating one or more embodiments of assessing a medical event in a patient according to one or more illustrative embodiments of the present disclosure. The method 100 includes positioning a virtual reality (VR) device on a patient to create a virtual environment (STEP 102); detecting one or more responses of the patient to stimulus introduced within the virtual environment (STEP 104); and determining whether the patient incurred a medical event based at least in part on the one or more responses (STEP 106). In certain embodiments, the medical event is a neurological event such as a cerebral stroke. In some embodiments, one or more STEPs may include instructing the patient to complete at least one of tests involving tracking or fixating on the stimulus within the virtual environment. Detecting one or more responses of the patient may include at least one of tracking or reaching movement toward the stimulus, the stimulus being one or more virtual objects introduced into the virtual environment. Tracking reaching movement toward the one or more virtual objects is performed in a first instance with the patient's hand is visible in the virtual environment and in a second instance when the patient's hand is not visible in the virtual environment. Determining whether the patient incurred a cerebellar stroke includes determining one of precision or accuracy of the subject relative to the virtual location of the target. In embodiments, fine motor movements of the subject (eye movement, reaching, or head movement) are detected. The present invention isolates those movements and measures their precision which may assist in determining the occurrence of the medical event, e.g., a stroke.

In some embodiments, tracking reaching movement toward the stimulus is performed in a first instance when the one or more virtual objects are visible in the virtual environment and in a second instance when the one or more virtual objects are not visible in the virtual environment. Determining whether the patient incurred a cerebral stroke includes determining one of precision or accuracy of the subject relative to the virtual location of the target.

In some embodiments, detecting one or more responses of the patient includes at least one of tracking eye movement; tracking truncal ataxia and tracking finger path and speed.

In some embodiments, the method includes quantifying the one or more responses to determine the occurrence of the medical event. For example, quantifying may include identifying or assigning precision metrics associated with reaching accuracies of the patient relative to the virtual targets. The data may be introduced into one or more tallying algorithms.

The method may optionally or further include initiating one of an intervention or treatment plan. (STEP 108) The method may optionally or further includes evaluating recovery by repeating one or more steps. (STEP 110).

### M. Conclusion

The acquisition of sensory information about the world is a dynamic and interactive experience, yet the majority of sensory research focuses on perception without action and is conducted with participants who are passive observers with very limited control over their environment. This approach allows for highly controlled, repeatable experiments and has led to major advances in our understanding of basic sensory processing.

Typical human perceptual experiences, however, are far more complex than conventional action-perception experiments and often involve bi-directional interactions between perception and action. Innovations in virtual reality (VR) technology offer an approach to close this notable disconnect between perceptual experiences and experiments. VR experiments can be conducted with a high level of empirical control while also allowing for movement and agency as well as controlled naturalistic environments. New VR technology also permits tracking of fine hand and eye movements, allowing for seamless empirical integration of perception and action. Here, we described published data using VR to assess how multisensory information and cognitive demands affect hand movements while reaching for virtual targets. First, we manipulated the visibility of the reaching hand to uncouple vision and proprioception in a task measuring accuracy while reaching toward a virtual target (n=20, healthy young adults). The results, which as expected revealed multisensory facilitation, provided a rapid and a highly sensitive measure of isolated proprioceptive accuracy. In the second experiment, we presented the virtual target only briefly and showed that VR can be used as an efficient and robust measurement of spatial memory (n=18, healthy young adults). Finally, to assess the feasibility of using VR to study perception and action in populations with physical disabilities, we showed that the results from the visual-proprioceptive task generalize to two patients with recent cerebellar stroke. Overall, we show that VR coupled with hand-tracking offers an efficient and adaptable way to study human perception and action.

This invention highlights the promising application of commercially available virtual reality headsets to efficiently study perceptual and motor processing during naturalistic eye, body, and hand movements. Assessment of cerebellar stroke involves clinical judgment of several factors, including those outlined in this proposal, that often relies on subjective evaluation of the patient until different conditions. The virtual assessment of these factors offers a rapid, objective alternative. Differences in reaching accuracy in various conditions were measurable in a short amount of time with very few trials, and the measures of eye movements and truncal stability are highly sensitive to small changes. By studying the action-perceptual loop in a dynamic, multisensory environment, the field of psychophysics can move closer to understanding how perception varies across real-life settings. The adaptability and mobility of this equipment also offers opportunities to uncouple visual and proprioceptive cues to study the weighting and interaction of these domains in clinical populations in any setting, as well as objectively measure eye and body movements under different conditions. As affordable and accessible technology, future work incorporating additional participant groups and multisensory environments offers great potential to understand how different factors affect sensory processing.

Intervention: In addition, by repeatedly doing the same hand-reaching assessment without a visually rendered hand (or with partial rendering) we can use this approach as a training intervention following stroke. The advance here is that we are able to isolate proprioceptive and motor processes while limiting the role of vision to guide these movements—thus allowing for an intervention that focuses on and isolates the motor system.

Assessment: Leveraging VR technology, we can rapidly and accurately assess eye and motor movement in patients who had strokes that affected their ocular or motor behavior, particularly upper-body coordination. A virtual reaching assessment accomplishes the same objective as the commonly used “finger-to-nose” assessment which is clinically used to track the course of stroke recovery, and virtual measurements of eye movements, truncal stability, and finger speed allow for rapid objective measurement of metrics that may narrow the differential diagnosis to that of a stroke. Our novel approach allows quantification of these behaviors with a major increase in accuracy, all taking only minutes.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

### N. References

1. Shamay-Tsoory S G, Mendelsohn A. Real-Life Neuroscience: An Ecological Approach to Brain and Behavior Research. Perspect Psychol Sci. 2019;14(5):841-59.
2. Holleman G A, Hooge I T C, Kemner C, Hessels R S. The ‘Real-World Approach’ and Its Problems: A Critique of the Term Ecological Validity. Front Psychol. 2020;11:721.
3. Jackson G M, Jackson S R, Kritikos A. Attention for action: coordinating bimanual reach-to-grasp movements. Br J Psychol. 1999;90(Pt 2 ) :247-70.
4. Naish K R, Reader A T, Houston-Price C, Bremner A J, Holmes N P. To eat or not to eat? Kinematics and muscle activity of reach-to-grasp movements are influenced by
   the action goal, but observers do not detect these differences. Experimental Brain Research. 2013;225(2):261-75.
5. Lang C E, Wagner J M, Bastian A J, Hu Q, Edwards D F, Sahrmann S A, et al. Deficits in grasp versus reach during acute hemiparesis. Experimental Brain Research. 2005;166(1):126-36.
6. Vercillo T, O'Neil S, Jiang F. Action-effect contingency modulates the readiness potential. Neuroimage. 2018;183:273-9.
7. Desantis A, Haggard P. How actions shape perception: learning action-outcome relations and predicting sensory outcomes promote audio-visual temporal binding. Sci Rep. 2016;6:39086.
8. Arikan B E, van Kemenade B M, Straube B, Harris L R, Kircher T. Voluntary and Involuntary Movements Widen the Window of Subjective Simultaneity. Iperception. 2017;8(4):2041669517719297.
9. Matthis J S, Yates J L, Hayhoe M M. Gaze and the Control of Foot Placement When Walking in Natural Terrain. Curr Biol. 2018;28(8):1224-33.e5.
10. Diaz G, Cooper J, Rothkopf C, Hayhoe M. Saccades to future ball location reveal memory-based prediction in a virtual-reality interception task. Journal of Vision. 2013;13(1):20.
11. Kothari R, Yang Z, Kanan C, Bailey R, Pelz J B, Diaz G J. Gaze-in-wild: A dataset for studying eye and head coordination in everyday activities. Scientific Reports. 2020;10(1):2539.
12. Hara M, Kanayama N, Blanke O, Salomon R. Modulation of Bodily Self-Consciousness by Self and External Touch. IEEE Trans Haptics. 2021;14(3):615-25.
13. Riemer M, Wolbers T, van Rijn H. Age-related changes in time perception: The impact of naturalistic environments and retrospective judgements on timing performance. Q J Exp Psychol (Hove). 2021;74(11):2002-12.
14. Voudouris D, Broda M D, Fiehler K. Anticipatory grasping control modulates somatosensory perception. J Vis. 2019;19(5):4.
15. Sonkusare S, Breakspear M, Guo C. Naturalistic Stimuli in Neuroscience: Critically Acclaimed. Trends Cogn Sci. 2019;23(8):699-714.
16. Tian F, Hua M, Zhang W, Li Y, Yang X. Emotional arousal in 2D versus 3D virtual reality environments. PLoS One. 2021;16(9): e0256211.
17. Knobel S E J, Kaufmann B C, Gerber S M, Cazzoli D, Müri R M, Nyffeler T, et al. Immersive 3D Virtual Reality Cancellation Task for Visual Neglect Assessment: A Pilot Study. Front Hum Neurosci. 2020;14:180.
18. Zhou Q, Hagemann G, Fafard D, Stavness I, Fels S. An Evaluation of Depth and Size Perception on a Spherical Fish Tank Virtual Reality Display. IEEE Trans VisComput Graph. 2019;25(5):2040-9.
19. Brinkman W P, Hoekstra A R, van Egmond R. The Effect Of 3D Audio And Other Audio Techniques On Virtual Reality Experience. Stud Health Technol Inform. 2015;219:44-8.
20. Rajguru C, Obrist M, Memoli G. Spatial Soundscapes and Virtual Worlds: Challenges and Opportunities. Front Psychol. 2020;11:569056.
21. Gehrke L, Akman S, Lopes P, Chen A, Singh A K, Chen H-T, et al. Detecting Visuo-Haptic Mismatches in Virtual Reality using the Prediction Error Negativity of Event-Related Brain Potentials. Proceedings of the 2019 CHI Conference on Human Factors in Computing Systems: Association for Computing Machinery; 2019. p. Paper 664 427.
22. Otaran A, Farkhatdinov I. Haptic Ankle Platform for Interactive Walking in Virtual Reality. IEEE Trans Vis Comput Graph. 2021;Pp.
23. McAnally K, Wallis G. Visual-haptic integration, action and embodiment in virtual reality. Psychol Res. 2021.
24. Cohen M A, Botch T L, Robertson C E. The limits of color awareness during active, real-world vision. Proc Natl Acad Sci U S A. 2020;117(24):13821-7.
25. Salanger M, Lewis D, Vallier T, McDermott T, Dergan A. Applying Virtual Reality to Audiovisual Speech Perception Tasks in Children. Am J Audiol. 2020;29(2):244-58.
26. Zaleski-King A, Pinto R, Lee G, Brungart D. Use of Commercial Virtual Reality Technology to Assess Verticality Perception in Static and Dynamic Visual Backgrounds. Ear Hear. 2020;41(1):125-35.
27. Zaki J, Ochsner K. The need for a cognitive neuroscience of naturalistic social cognition. Ann N Y Acad Sci. 2009;1167:16-30.
28. Nijman S A, Veling W, Greaves-Lord K, Vos M, Zandee C E R, Aan Het Rot M, et al. Dynamic Interactive Social Cognition Training in Virtual Reality (DiSCoVR) for People With a Psychotic Disorder: Single-Group Feasibility and Acceptability Study. JMIR Ment Health. 2020;7(8):e17808.
29. Park J, Son B, Han I, Lee W. Effect of Cutaneous Feedback on the Perception of Virtual Object Weight during Manipulation. Sci Rep. 2020;10(1):1357.
30. Weech S, Kenny S, Barnett-Cowan M. Presence and Cybersickness in Virtual Reality Are Negatively Related: A Review. Front Psychol. 2019;10:158.
31. Smith S A, Mulligan N W. Immersion, presence, and episodic memory in virtual reality environments. Memory. 2021;29(8):983-1005.
32. Aoyagi K, Wen W, An Q, Hamasaki S, Yamakawa H, Tamura Y, et al. Modified sensory feedback enhances the sense of agency during continuous body movements in virtual reality. Sci Rep. 2021;11(1):2553.
33. Caruana N, Spirou D, Brock J. Human agency beliefs influence behaviour during virtual social interactions. PeerJ. 2017;5:e3819.
34. Peelen M V, Kastner S. Attention in the real world: toward understanding its neural basis. Trends Cogn Sci. 2014;18(5):242-50.
35. Eger Passos D, Jung B, editors. Measuring the Accuracy of Inside-Out Tracking in XR Devices Using a High-Precision Robotic Arm2020;Cham: Springer International Publishing.
36. Thielbar K O, Triandafilou K M, Barry A J, Yuan N, Nishimoto A, Johnson J, et al. Home-based Upper Extremity Stroke Therapy Using a Multiuser Virtual Reality Environment: A Randomized Trial. Arch Phys Med Rehabil. 2020;101(2):196-203.
37. Chen Y, Abel K T, Janecek J T, Chen Y, Zheng K, Cramer S C. Home-based technologies for stroke rehabilitation: A systematic review. Int J Med Inform. 2019;123:11-22.
38. Truijen S, Abdullahi A, Bijsterbosch D, van Zoest E, Conijn M, Wang Y, et al. Effect of home-based virtual reality training and telerehabilitation on balance in individuals with Parkinson disease, multiple sclerosis, and stroke: a systematic review and meta-analysis. Neurol Sci. 2022;43(5):2995-3006.
39. Kairy D, Veras M, Archambault P, Hernandez A, Higgins J, Levin M F, et al. Maximizing post-stroke upper limb rehabilitation using a novel telerehabilitation interactive virtual reality system in the patient's home: study protocol of a randomized clinical trial. Contemp Clin Trials. 2016;47:49-53.
40. Piron L, Turolla A, Agostini M, Zucconi C, Cortese F, Zampolini M, et al. Exercises for paretic upper limb after stroke: a combined virtual-reality and telemedicine approach. J Rehabil Med. 2009;41(12): 1016-102.
41. Schröder J, van Criekinge T, Embrechts E, Celis X, Van Schuppen J, Truijen S, et al. Combining the benefits of tele-rehabilitation and virtual reality-based balance training: a systematic review on feasibility and effectiveness. Disabil Rehabil Assist Technol. 2019;14(1):2-11.
42. Lin R C, Chiang S L, Heitkemper M M, Weng S M, Lin C F, Yang F C, et al. Effectiveness of Early Rehabilitation Combined With Virtual Reality Training on Muscle Strength, Mood State, and Functional Status in Patients With Acute Stroke: A Randomized Controlled Trial. Worldviews Evid Based Nurs. 2020;17(2):158-67.
43. Patel J, Fluet G, Qiu Q, Yarossi M, Merians A, Tunik E, et al. Intensive virtual reality and robotic based upper limb training compared to usual care, and associated cortical reorganization, in the acute and early sub-acute periods post-stroke: a feasibility study. J Neuroeng Rehabil. 2019;16(1):92.
44. Park Y-S, An C-S, Lim C-G. Effects of a Rehabilitation Program Using a Wearable Device on the Upper Limb Function, Performance of Activities of Daily Living, and Rehabilitation Participation in Patients with Acute Stroke. Int J Environ Res Public Health. 2021;18(11):5524.
45. In T, Lee K, Song C. Virtual Reality Reflection Therapy Improves Balance and Gait in Patients with Chronic Stroke: Randomized Controlled Trials. Med Sci Monit. 733 2016;22:4046-53.
46. Lee H S, Park Y J, Park S W. The Effects of Virtual Reality Training on Function in Chronic Stroke Patients: A Systematic Review and Meta-Analysis. Biomed Res Int. 736 2019;2019:7595639.
47. Kiper P, Szczudlik A, Agostini M, Opara J, Nowobilski R, Ventura L, et al. Virtual Reality for Upper Limb Rehabilitation in Subacute and Chronic Stroke: A Randomized Controlled Trial. Arch Phys Med Rehabil. 2018;99(5):834-42.e4.
48. Yin C W, Sien N Y, Ying L A, Chung S F, Tan May Leng D. Virtual reality for upper extremity rehabilitation in early stroke: a pilot randomized controlled trial. Clin Rehabil. 2014;28(11):1107-14.
49. Mekbib D B, Debeli D K, Zhang L, Fang S, Shao Y, Yang W, et al. A novel fully immersive virtual reality environment for upper extremity rehabilitation in patients with stroke. Ann N Y Acad Sci. 2021;1493(1):75-89.
50. Kim W-S, Cho S, Park S H, Lee J-Y, Kwon S, Paik N-J. A low cost kinect-based virtual rehabilitation system for inpatient rehabilitation of the upper limb in patients with subacute stroke: A randomized, double-blind, sham-controlled pilot trial. Medicine (Baltimore). 2018;97(25):e11173-e.
51. O'Brien A T, Bertolucci F, Torrealba-Acosta G, Huerta R, Fregni F, Thibaut A. Non-invasive brain stimulation for fine motor improvement after stroke: a meta-analysis. Eur J Neurol. 2018;25(8):1017-26.
52. Ivry R B, Keele S W, Diener H C. Dissociation of the lateral and medial cerebellum in movement timing and movement execution. Exp Brain Res. 1988;73(1):167-80.
53. Holmes N P, Crozier G, Spence C. When mirrors lie: “visual capture” of arm position impairs reaching performance. Cogn Affect Behav Neurosci. 2004;4(2):193-757 200.
54. Bernier P-M, Chua R, Franks I M, Khan M A. Determinants of Offline Processing of Visual Information for the Control of Reaching Movements. Journal of Motor Behavior. 2006;38(5):331-8.
55. Caplan B, Mendoza J E. Edinburgh Handedness Inventory. In: Kreutzer J S, DeLuca J, Caplan B, editors. Encyclopedia of Clinical Neuropsychology. New York, NY: Springer New York;2011. p. 928-.
56. Prime S L, Marotta J J. Gaze strategies during visually-guided versus memory-guided grasping. Exp Brain Res. 2013;225(2):291-305.
57. Heath M, Westwood D A, Binsted G. The control of memory-guided reaching movements in peripersonal space. Motor Control. 2004;8(1):76-106.
58. Reichenbach A, Thielscher A, Peer A, Bülthoff H H, Bresciani J P. Seeing the hand while reaching speeds up on-line responses to a sudden change in target position. J Physiol. 2009;587(Pt 19):4605-16.
59. Sober S J, Sabes P N. Flexible strategies for sensory integration during motor planning. Nat Neurosci. 2005;8(4):490-7.
60. Westwood D A, Heath M, Roy E A. The accuracy of reaching movements in brief delay conditions. Can J Exp Psychol. 2001;55(4):304-10.
61. Admiraal M A, Keijsers N L, Gielen C C. Interaction between gaze and pointing toward remembered visual targets. J Neurophysiol. 2003;90(4):2136-48.
62. Snijders H J, Holmes N P, Spence C. Direction-dependent integration of vision and proprioception in reaching under the influence of the mirror illusion. Neuropsychologia. 2007;45(3):496-505.
63. Sexton B M, Liu Y, Block H J. Increase in weighting of vision vs. proprioception associated with force field adaptation. Sci Rep. 2019;9(1):10167.
64. Wann J P. The integrity of visual-proprioceptive mapping in cerebral palsy. Neuropsychologia. 1991;29(11):1095-106.
65. Goodworth A, Saavedra S. Postural mechanisms in moderate-to-severe cerebral palsy. J Neurophysiol. 2021;125(5):1698-719.
66. Feller K J, Peterka R J, Horak F B. Sensory Re-weighting for Postural Control in Parkinson's Disease. Frontiers in Human Neuroscience. 2019;13.
67. Tagliabue M, Ferrigno G, Horak F. Effects of Parkinson's disease on proprioceptive control of posture and reaching while standing. Neuroscience. 2009;158(4):1206-14.
68. Lidstone D E, Mostofsky S H. Moving Toward Understanding Autism: Visual-Motor Integration, Imitation, and Social Skill Development. Pediatric Neurology. 2021;122:98-105.
69. Sharer E A, Mostofsky S H, Pascual-Leone A, Oberman L M. Isolating Visual and Proprioceptive Components of Motor Sequence Learning in ASD. Autism Res. 2016;9(5):563-9.
70. Diersch N, Wolbers T. The potential of virtual reality for spatial navigation research across the adult lifespan. Journal of Experimental Biology. 799 2019;222(Suppl_1):jeb187252.
71. Caglio M, Latini-Corazzini L, D'Agata F, Cauda F, Sacco K, Monteverdi S, et al. Virtual navigation for memory rehabilitation in a traumatic brain injured patient. Neurocase. 2012;18(2):123-31.
72. Pahor A, Collins C, Smith R N, Moon A, Stavropoulos T, Silva I, et al. Multisensory Facilitation of Working Memory Training. J Cogn Enhanc. 2021;5(3):386-
73. Shams L, Seitz A R. Benefits of multisensory learning. Trends Cogn Sci. 2008;12(11):411-7.
74. Honoré J, Bourdeaud'hui M, Sparrow L. Reduction of cutaneous reaction time by directing eyes towards the source of stimulation. Neuropsychologia. 1989;27(3):367-71.
75. Rodrigues M R, Slimovitch M, Chilingaryan G, Levin M F. Does the Finger-to-Nose Test measure upper limb coordination in chronic stroke? J Neuroeng Rehabil. 2017;14(1):6.
76. National Institute of Neurological D, Stroke. NIH stroke scale: [Bethesda, Md.] National Institute of Neurological Disorders and Stroke, Dept. of Health and Human Services, USA, [2011?];2011.

What is claimed is:

1. A method of rapid assessment of eye, head, and hand reaching movements using virtual reality and application in cerebellar stroke comprising:
providing a VR device to be worn by a stroke patient;
instructing the stroke patient to track or fixate on targets with their eyes, passively measure truncal stability, measure the speed and regularity of finger tapping, reach for a VR target without a view of their own hand while reaching for the target, forcing a high reliance on proprioception; and
evaluating a reaching error in reaching for the target based on a reach accuracy.

2. The method of claim 1, further comprising a treatment or intervention by instructing a repeated performance of the steps instructing through evaluating.

3. The method of claim 1 further comprising sounding an auditory cue which serves as a cue for the participant to reach for the target location.

4. A method for assessing a patient, comprising:
positioning a virtual reality (VR) device on a patient to create a virtual environment;
detecting one or more responses of the patient to stimulus introduced within the virtual environment; and
determining whether the patient incurred a medical event based at least in part on the one or more responses.

5. The method of claim 4 wherein the medical event is a neurological event.

6. The method of claim 5 wherein the neurological event is a cerebral stroke.

7. The method of claim 6 including instructing the patient to at least one of track or fixate on the stimulus within the virtual environment.

8. The method of claim 7 wherein detecting one or more responses of the patient includes at least one of tracking reaching movement toward the stimulus, the stimulus being one or more virtual objects introduced into the virtual environment.

9. The method of claim 8 wherein tracking reaching movement toward the one or more virtual objects is performed in a first instance with the patient's hand visible in the virtual environment and in a second instance when the patient's hand is not visible in the virtual environment.

10. The method of claim 9 wherein determining whether the patient incurred a cerebral stroke includes determining differences in one or more of precision or accuracy of the tracked reaching movements during the first instance and the second instance.

11. The method of claim 8 wherein tracking reaching movement toward the stimulus is performed in a first instance when the one or more virtual objects are visible in the virtual environment and in a second instance when the one or more virtual objects are not visible in the virtual environment.

12. The method of claim 11 wherein determining whether the patient incurred a cerebellar stroke determining differences in one or more of precision or accuracy of the tracked reaching movements during the first instance and the second instance.

13. The method of claim 7 wherein detecting one or more responses of the patient includes at least one of tracking eye movement; tracking truncal ataxia and tracking finger speed.

14. The method of claim 4 including quantifying the one or more responses to determine the occurrence of the medical event.

15. The method of claim 4 including initiating one of an intervention or treatment plan.

16. The method of claim 15 further including evaluating recovery by repeating one or more steps.

17. A method for assessing a patient, comprising:
positioning a virtual reality (VR) device on a patient to create a virtual environment; and
measuring the patient's fine motor movements in one or more responses of the patient to stimulus introduced within the virtual environment to determine whether the patient incurred a medical event.

18. The method of claim 17 wherein the medical event is one of a neurological event or a cerebellar stroke.
